(19) United States
(12) Patent Application Publication
(10) Pub. No.: US 2018/0185236 A1
(43) Pub. Date: Jul. 5, 2018

(54) MULTIFUNCTIONAL FACIAL AND BODY TREATMENT DEVICE

(71) Applicant: BenZion Levi, Habonim (IL)

(72) Inventor: BenZion Levi, Habonim (IL)

(21) Appl. No.: 15/398,909

(22) Filed: Jan. 5, 2017

(51) Int. Cl. A61H 15/02; A46B 13/02; A46B 13/00; A46B 5/00; A46B 15/00; A47K 7/04; A61N 1/04; A61N 1/40; A61N 5/06; A61N 1/26

(52) U.S. Cl. CPC A61H 15/02; A61N 2005/0652; A46B 13/008; A46B 5/0095; A46B 15/0014; A46B 15/0022; A46B 15/003; A46B 15/0036; A47K 7/04; A61N 1/0476; A61N 1/40; A61N 5/0616; A61N 1/26; A61H 2201/10; A61H 2201/0207; A61H 2201/0214; A61H 2201/0285; A46B 13/02

(57) ABSTRACT

A multifunctional treatment device includes a housing, a processing unit, a power driver unit, and a motor configured to rotate a brush interface element. RF electrodes and electrical terminals are connected to the power driver unit. A plurality of treatment heads are connectable to the housing. One of the treatment heads includes a brush head with bristles and one or more non-conductive barriers. The brush head is formed with apertures and the RF electrodes pass through and protrude out of the apertures. Another of the treatment heads includes a movable brush head. Another one of the stationary treatment heads includes a treatment surface configured to contact a skin or body of a user and one or more active components for performing different kinds of treatment.

### FIELD OF THE INVENTION

The present invention relates generally to facial and body treatment devices, and particularly to multifunctional facial and body treatment device with brush elements and RF electrodes.

### BACKGROUND OF THE INVENTION

Many facial and body treatment devices are available on the market that use brushes to remove unwanted substances and particles from the user's skin or body.

### SUMMARY OF THE INVENTION

The present invention seeks to provide an improved multifunctional facial and body treatment device with brush elements, treatment heads, electrical terminals and RF electrodes, as is described more in detail hereinbelow.

There is thus provided in accordance with an embodiment of the present invention a multifunctional treatment device including a housing with one or more control buttons, a processor, a power driver unit in communication with the processor and operatively connected to a motor configured to rotate a brush interface element, RF electrodes and electrical terminals connected to the power driver unit, and a plurality of treatment heads connectable to the housing, wherein one of the treatment heads includes a brush head that includes bristles and one or more non-conductive barriers, the brush head not being connected to the motor, the brush head being formed with apertures, wherein the RF electrodes are arranged to pass through and protrude out of the apertures, and wherein another one of the treatment heads includes a movable brush head that includes bristles and which is connectable to the motor; and wherein another one of the treatment heads includes a treatment surface configured to contact a skin or body of a user and one or more active components for performing different kinds of treatment.

The inner surfaces of the treatment head may include one or more communication components such as NFC (Near Field Communication).

The treatment surface may be smooth or may include treatment elements, such as massage rollers for face and neck massage. A return electrode may also be provided that works in cooperation with electrodes for use in various treatments, such as enhancing absorption of skin products (such as with negatively charged ions), softening skin (such as with positively charged ions), or facial toning.

The active components may include thermoelectric (Peltier effect) elements for heating or cooling the treatment surface.

### BRIEF DESCRIPTION OF THE DRAWINGS

The present invention will be understood and appreciated more fully from the following detailed description, taken in conjunction with the drawings in which:

FIGS. 1 and 2 are schematic illustrations of a multifunctional (facial and body) treatment device, in accordance with a non-limiting embodiment of the invention, respectively with a brush head attached to and removed from the housing of the device.

FIG. 3 is a schematic illustration of the device with a different brush head.

FIG. 4 is a schematic illustration of the device with an active brush head for skin treatment, according to another non-limiting embodiment of the invention.

FIGS. 5A-5E are schematic illustrations of different treatment heads for use with the device of FIG. 1, according to additional non-limiting embodiments of the invention.

FIGS. 6A and 6B are simplified perspective illustrations of the device of FIG. 1, showing electrodes and a non-conductive barrier between them, according to an additional non-limiting embodiment of the invention.

FIG. 7 is a simplified illustration of the bristle head portion, electrodes and non-conductive barrier between them, according to a non-limiting embodiment of the invention.

### DETAILED DESCRIPTION OF EMBODIMENTS

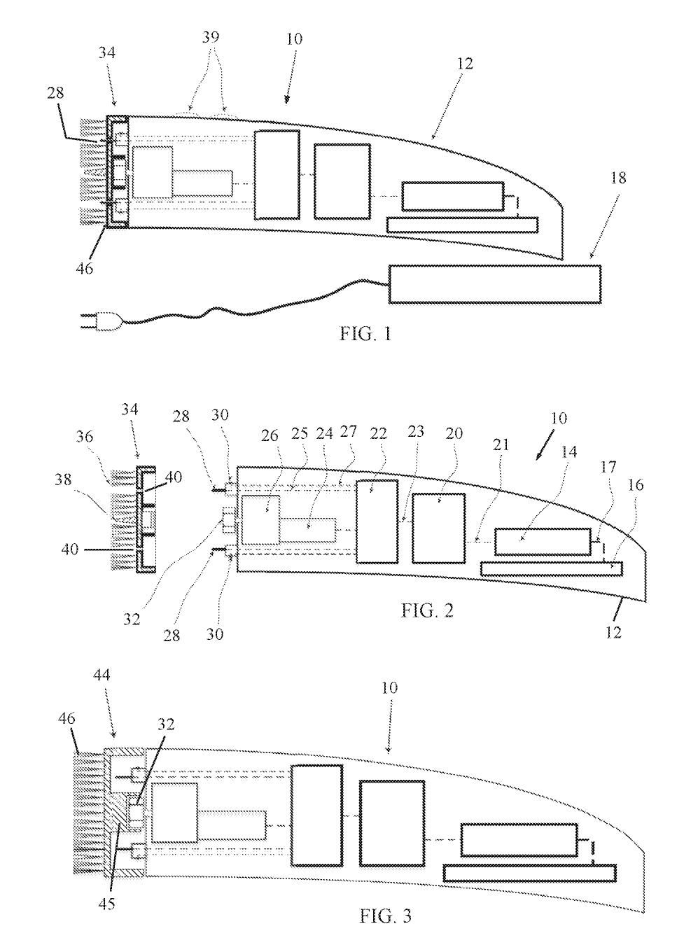

Reference is now made to FIGS. 1 and 2, which illustrate a multifunctional facial and body treatment device 10, constructed and operative in accordance with a non-limiting embodiment of the present invention.

Device 10 includes a housing 12 in which operating components of the device are housed. All of the electrical components may be mounted on a printed circuit board and operated with one or more control buttons 39 (of any size and shape, shown in FIG. 1). The operating components include a power source 14, such as one or more rechargeable batteries, which may be recharged by means of a charger module 16, connected by electrical connection 17 to battery or batteries 14, and which may be in wireless communication with an external wireless charging unit 18 (shown in FIG. 1). Of course, power source 14 may be recharged by wired connection and instead does not have to be rechargeable.

Device 10 includes a processing unit 20, connected by electrical connection 21 to power source 14. The processing unit 20 may be connected by electrical connection 23 to a power driver unit 22 (which, for example, may include power amplification circuitry, current regulation, microcurrent source, radio-frequency (RF) generation circuitry, and others), which drives a DC motor 24 connected to or part of a motor 26, such as a step motor or gear motor.

A plurality of RF electrodes 28 are connected by RF electrode connections 25 to power driver unit 22. A plurality of electrical terminals 30 are connected by electrical connection 27 to power driver unit 22.

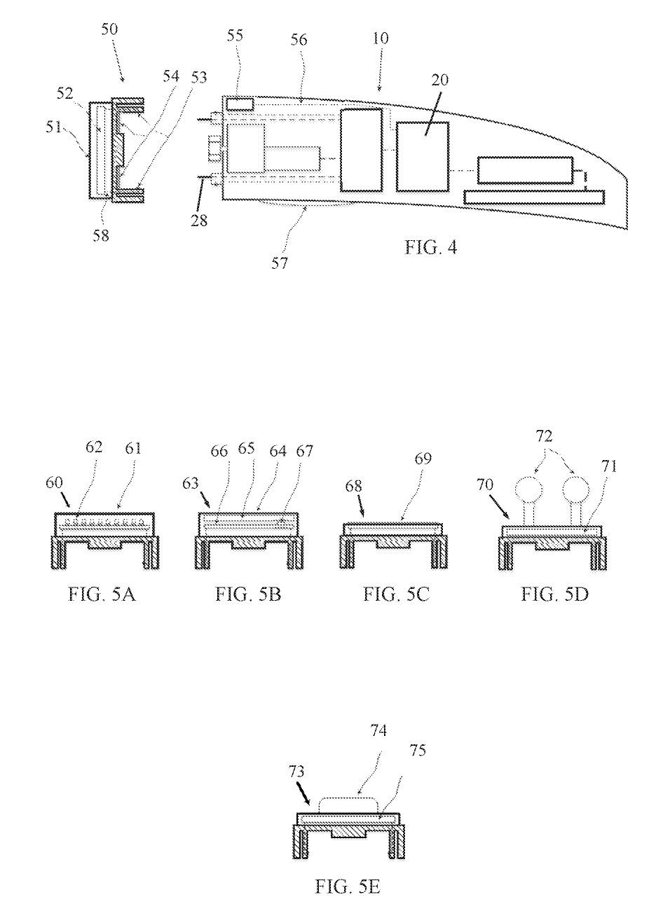

Gear motor 26 rotates a brush interface element 32, such as a hub, lug or other suitable element, to which a user can attach a treatment head unit, such as a brush head 34 (also referred to as treatment head 34), which includes bristles 36 and one or more non-conductive barriers 38. Brush head 34 may be used for cleansing and skin rejuvenation treatments, for example. Another brush head 50 (shown in FIG. 4) may be stationary, that is, not connected to brush interface element 32.

The RF generator of power driver unit 22 may generate RF energy in a frequency range, typically but not limited to, 500 KHz-30 MHz. The power driver unit 22 may generate galvanic energy in a current range, typically but not limited to 50 microamperes to 250 microamperes and frequency range, typically but not limited to, 1 Hz-500 Hz. The galvanic energy delivered through an electrode 69 (shown in FIG. 5C) placed on a treatment head and returns through a returned electrode 57 (shown in FIG. 4).

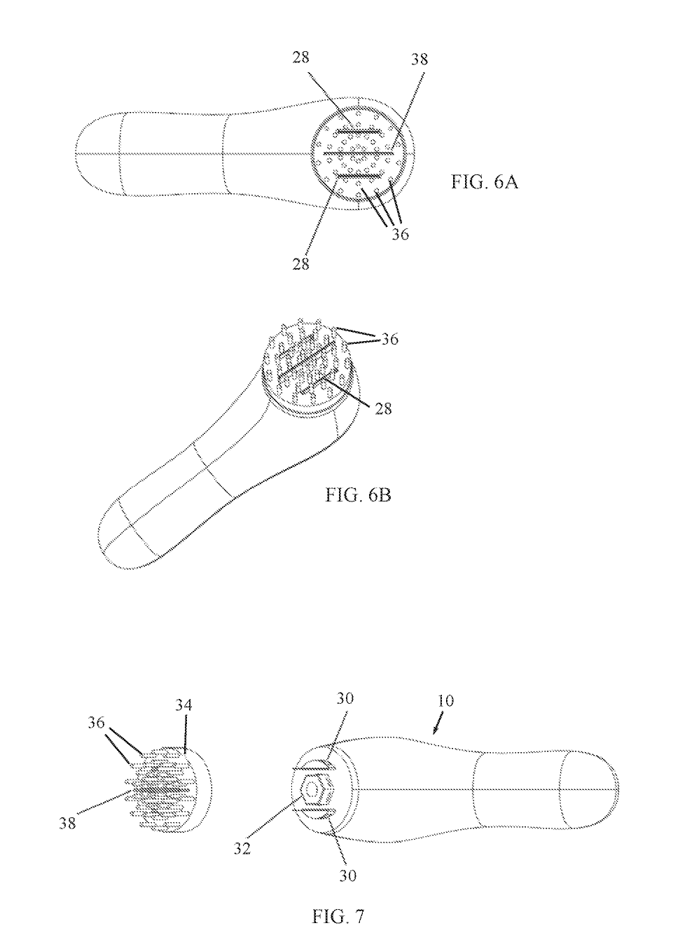

Reference is now made additionally to FIGS. 6A, 6B and 7, which illustrate brush head 34 with bristles 36, RF electrodes 28 and non-conductive barriers 38 between them.

Brush head 34 may be formed with apertures 40. When brush head 34 is mounted on housing 12, the RF electrodes 28 pass through and protrude out of apertures 40.

The RF electrodes 28 serve as different RF poles, which when placed against a user's skin, may make contact with an electrolyte present on the skin (e.g., cream, lotion, salve, etc.) and thereby generate or release chemical agents at the application site.

Many configurations of the RF poles 28 and bristles (also referred to as cleaning elements) 36 of the brush head can be made in accordance with the invention. For example, bristles 36 may be located between the non-conductive barriers 38 and each of the electrodes 28, or just between some of the non-conductive barriers 38 and some of the electrodes 28 or other configurations.

In this embodiment, as well as any of the other embodiments, a temperature sensor 43 may be located on the brush head 34 and/or the non-conductive barrier 38. The temperature sensor 43 may be in communication with the processor 20 for controlling the operation of the RF generator. If the temperature rises above a non-safe temperature (e.g., 41° C.), then the sensor 43 turns off the RF energy.

The electrodes 28 and/or the bristles 36 may have different heights and thicknesses to create different electrical and electromagnetic fields in the vicinity of the brush head 34 and barrier 38.

Reference is now made to FIG. 3, which illustrates another brush head 44 mounted on multifunctional facial and body treatment device 10. Brush head 44 includes bristles 46 and may be used for cleansing treatments, for example. Brush head 44 may be a movable head that is connected to gear motor 26 by means of a socket 45 that mates with brush interface element 32 of gear motor 26. In this manner, brush head 44 is rotated in any direction or chosen speed.

Reference is now made to FIG. 4, which illustrates a treatment head 50 that includes a treatment surface 51 configured to contact the skin or body of a user. Inside treatment head 50, behind treatment surface 51, are located active components 52 for performing different kinds of treatment (examples of which are given with reference to FIGS. 5A-5E). The treatment surface 51 may be smooth or may include treatment elements, such as massage rollers for face and neck massage. The inner surfaces of treatment head 50 may include one or more electrical contacts 53, identification components 54, such as RF tags, and other components. The RF tag may cooperate with an RF reader/writer (e.g., NFC) 55 connected by connection 56 to processing unit 20. A return electrode 57 may also be provided that works in cooperation with electrodes 69 for use in various treatments, such as enhancing absorption of skin products (such as with negatively charged ions), softening skin (such as with positively charged ions), or facial toning.

Reference is now made to FIGS. 5A-5E, which illustrate different treatment heads. In FIG. 5A, a treatment head 60 has a transparent treatment surface 61 and the active components include LEDs 62. Treatment head 61 may be used, without limitation, for skin rejuvenation or acne treatment.

In FIG. 5B, a treatment head 63 has a treatment surface 64 and the active components may include thermoelectric (Peltier effect) elements 65 for heating or cooling treatment surface 64, driver circuitry 66 for driving and controlling the thermoelectric elements 65 and a temperature sensor 67 as a safety measure, as described above.

Treatment head 63 may be used, without limitation, for detoxification and shrinkage of pores (with treatment surface 64 being cooled, such as to 5° C. by the thermoelectric element 65), or for opening of pores for deep cleaning (with treatment surface 64 being heated, such as to 40° C. by the thermoelectric element 65).

In FIG. 5C, a treatment head 68 has a conductive plate 69 in electric communication with return electrode 57.

In FIG. 5D, a treatment head 70 may include a microcurrent generator 71 connected to electrodes 72. Treatment head 70 may be used for facial toning or skin rejuvenation, for example. The microcurrent source, the conductive surface and the electrodes can operate in bipolar or galvanic modes or a combination thereof. The electrodes 72 may also be connected to the power driver unit 22 for generating RF energy,

In FIG. 5E, a treatment head 73 has a treatment surface 74, and active components may include a piezoelectric generator 75. Treatment head 73 may be used as a beauty massager.

What is claimed is:

1. A multifunctional treatment device comprising:
a housing with one or more control buttons;
a processing unit;
a power driver unit in communication with said processing unit and operatively connected to a motor configured to rotate a brush interface element;
RF electrodes and electrical terminals connected to said power driver unit; and
a plurality of treatment heads connectable to said housing, wherein one of said treatment heads comprises a brush head that comprises bristles and one or more non-conductive barriers, said brush head not being connected to said motor, said brush head being formed with apertures, wherein said RF electrodes are arranged to pass through and protrude out of said apertures,
and wherein another one of said treatment heads comprises a movable brush head that comprises bristles and which is connectable to said motor;
and wherein another one of said treatment heads comprises a treatment surface configured to contact a skin or body of a user and one or more active components for performing different kinds of treatment.

2. The multifunctional treatment device according to claim 1, wherein said treatment surface is smooth.

3. The multifunctional treatment device according to claim 1, wherein said treatment surface comprises one or more rollers.

4. The multifunctional treatment device according to claim 1, wherein inner surfaces of said treatment head comprise one or more electrical contacts.

5. The multifunctional treatment device according to claim 1, wherein inner surfaces of said treatment head comprise one or more identification components.

6. The multifunctional treatment device according to claim 1, wherein said treatment surface is transparent.

7. The multifunctional treatment device according to claim 1, wherein at least one of said active components comprises an LED.

8. The multifunctional treatment device according to claim 1, wherein at least one of said active components comprises a thermoelectric element for heating or cooling said treatment surface.

9. The multifunctional treatment device according to claim 1, wherein said housing comprises a temperature sensor.

10. The multifunctional treatment device according to claim 1, wherein said treatment head comprises a conductive plate in electric communication with a return electrode.

11. The multifunctional treatment device according to claim 1, wherein said treatment head comprises a microcurrent generator and microcurrent electrodes.

12. The multifunctional treatment device according to claim 1, further comprising RF electrodes, and wherein said treatment head is connected to said power driver unit for generating RF energy with said RF electrodes.

13. The multifunctional treatment device according to claim 1, wherein said treatment head comprises a piezoelectric generator and a treatment surface.
